(19) United States
(12) Patent Application Publication
(10) Pub. No.: US 2007/0184138 A1
(43) Pub. Date: Aug. 9, 2007

(54) Method of preparing a cosmetic composition, and an apparatus for implementing such a method

(75) Inventors: Roland De La Mettrie, Le Vesinet (FR); Jean-Francois Grollier, Paris (FR); Jean Cotteret, Verneuil Sur Seine (FR)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/698,178

(22) Filed: Jan. 26, 2007

Related U.S. Application Data
(60) Provisional application No. 60/773,315, filed on Feb. 15, 2006.

(30) Foreign Application Priority Data
Jan. 27, 2006 (FR) 06 50304

(51) Int. Cl. A61K 8/02; A61K 8/00

(52) U.S. Cl. 424/769; 424/401; 424/750; 424/70.2

(57) ABSTRACT

A method of preparing a cosmetic composition includes injecting into a plurality of enclosures containing different substances at least one fluid via a plurality of injectors. The method produces a plurality of solutions which, after mixing, form all or part of the cosmetic composition.

### CROSS REFERENCE TO RELATED APPLICATIONS

This non provisional application claims the benefit of French Application No. 06 50304 filed on Jan. 27, 2006 and U.S. Provisional Application No. 60/773,315 filed on Feb. 15, 2006, the entire disclosure of which is incorporated by reference herein.

The present invention relates to cosmetic compositions, and more particularly to those obtained by causing a fluid to flow in contact with a substance having at least one compound that can be extracted by the fluid.

### BACKGROUND

The following European patent applications: EP 1 563 827-A2, EP 1 566 164-A1, EP 1 563 885-A1, EP 1 559 400-A1, EP 1 559 398-A1, EP 1 563 826-A1, EP 1 559 414-A1, EP 1 563 826-A1, EP 1 559 401-A1, EP 1 559 392-A1, EP 1 559 396-A1, and EP 1 556 163-A1, all disclose methods of preparing a cosmetic composition by percolating a fluid.

Application WO 00/56629 discloses a cartridge for an apparatus for extraction by percolation.

### SUMMARY

There exists a need to improve methods of preparing cosmetic compositions.

There also exists a need to prepare personalized cosmetic compositions in a manner that is reliable and relatively simple, and to facilitate the packaging and use of substances involved in preparing such compositions.

The invention seeks to satisfy these needs in full or in part.

Thus, in one of its aspects, the invention provides a method of preparing a cosmetic composition, the method comprising:

injecting into a plurality of enclosures containing different substances at least one fluid by using a plurality of injectors so as to produce a plurality of solutions which, after mixing, form all or part of the cosmetic composition.

Compared to using a single injector, the use of a plurality of injectors can reduce the risk of an enclosure becoming contaminated by a substance coming from another enclosure.

This can possibly also result in a simplification of the operations that need to be performed in order to prepare the cosmetic composition, and in particular it can result in a saving in time.

The fluid may be injected into a plurality of enclosures by using injectors that are respectively associated with different enclosures of the plurality of enclosures.

The enclosure may be a compartment, in particular a compartment of a cartridge.

Embodiments of the invention can facilitate preparing a cosmetic composition from a plurality of substances contained in different enclosures. Embodiments of the invention can make it possible to prepare a personalized composition by selecting the enclosures in which injection is to take place.

The method may comprise selecting at least one desired property of the cosmetic composition or at least one level for a given property of the cosmetic composition, and controlling the injection of fluid in such a manner as to obtain the selected property and/or its anticipated level. The selected property may relate to the color of the composition or to its dyeing capacity. Embodiments of the invention can also make it easier to package and store substances used for preparing the composition, by making it possible to use substances that are not liquids.

Finally, embodiments of the invention can make it easier to use substances of natural origin, in particular plant extracts or minerals.

The flow of fluid through the enclosure(s) may be by percolation.

The term “percolation” designates passing a fluid through a medium that is permeable to the fluid, the medium being in powder form, for example. The medium may be dissolved entirely by the passage of the fluid, where appropriate. The medium need not be a foodstuff.

The fluid, which is sometimes also known as a percolate, may comprise a liquid and/or a gas, e.g. water. The fluid may be water. Other solvents can be used, e.g. a solution in alcohol or in oil.

The injectors may serve to inject the same fluid or different fluids.

The quantity of substance contained in an enclosure may be relatively small, e.g. corresponding to a single use. The quantity of substance may be less than or equal to 25 cubic centimeters (cm³), or 10 cm³, or 7.5 cm³, or indeed 5 cm³, for example.

Once extraction has been performed by the fluid, the corresponding enclosure can be disposed of, e.g. by being recycled.

The enclosures disposed on the extractor apparatus may optionally belong to a single refill.

The fluid may flow simultaneously or otherwise through the various enclosures of a plurality of enclosures.

Where appropriate, each injector may be subjected, between the fluid injection steps, to cleaning, e.g. by a flow of fluid in and/or over the injector. The injectors may also be removable in order to make them easier to clean.

At least one of the enclosures may include an agent for coloring keratinous materials, e.g. the skin, the lips, the hair, and other keratinous fibers such as the eyelashes and the eyebrows.

Different enclosures in a single refill may contain different coloring agents, or the same coloring agents but at different concentrations.

The same refill may include coloring agents that enable a plurality of shades of a given color to be obtained.

The coloring agent(s) may be for performing dyeing directly, or by oxidation, optionally for bleaching purposes. For example, it may constitute tone on tone coloring.

The coloring agent(s) may be direct dyes or oxidation dyes.

Injection may be performed in at least one enclosure selected from a plurality of enclosures.

Prior to selecting at least one enclosure, at least one item of data may be acquired that is associated with the person to be treated with the cosmetic composition, and the enclosure(s) may be selected on the basis of the at least one item of data.

The data may comprise a color, e.g. that of the skin or the hair of the person to be treated with the composition.

By way of example, the enclosures may be selected to reproduce a tint of the skin or of the hair, and it is also possible for selection to be for the purpose of reproducing a color selected by a user from a color chart or a hair color chart or corresponding to a commercial reference, for example.

The substances contained in the enclosures may be in powder form.

Where appropriate, a single enclosure may contain at least two compounds in powder form. These two compounds may be mixed, optionally uniformly, within the enclosure. By way of example, the compounds may be placed in the enclosure in successive layers.

The temperature of the fluid injected into the enclosure may be greater than 80° C., thus encouraging certain compounds to dissolve, the fluid possibly being in the form of both vapor and liquid phases, thus facilitating extraction.

The injection pressure is preferably greater than or equal to 1 bar (10⁵ Pa), or greater than or equal to 3 bars, or more preferably greater than or equal to 10 bars, e.g. being as high as 10 bars to 50 bars.

The solution(s) coming from the extraction may be collected in a receptacle, e.g. in a bowl.

The receptacle may optionally be provided with a closure member, in particular for stirring purposes, and/or to enable the consumer to take the composition for use away from the premises on which it was prepared.

The receptacle may optionally contain at least one compound for mixing with the solution(s) obtained by the extraction, in particular a cosmetically-acceptable medium, e.g. a base or a dye oxidizer.

Each of the enclosures may be defined at least in part by a first wall suitable for having at least one injector of the extractor apparatus pass therethrough, e.g. by being perforated.

Each of the enclosures may also be defined at least in part by a second wall suitable for opening under the effect of the pressure at which the fluid is injected into the enclosure, e.g. by the second wall being deformed against at least one portion in relief that is suitable for causing it to open. By way of example, the second wall is deformed against a grid arranged so that deformation is accompanied by the enclosure opening, while also serving to enable the second wall to retain substantially all of the substance that has not been dissolved by the fluid flowing through the enclosure.

By way of example, the first and second walls comprise at least one layer of material that is suitable for being tom and/or perforated relatively easily, e.g. a layer comprising a metal, e.g. aluminum, of thickness less than or equal to 0.1 millimeters (mm), for example.

In another of its aspects, the invention also provides an assembly enabling a cosmetic composition to be prepared, the assembly comprising:

a plurality of enclosures containing different cosmetic substances; and

an extractor apparatus comprising a plurality of injectors in order to inject at least one fluid into at least one enclosure and collect at least one solution by the fluid flowing through the enclosure.

It is possible to use only one injector at a time, or in a variant, a plurality of injectors can be used at the same time for injecting the fluid. By way of example, the use of a single injector can make it easier to respect an injection pressure and can simplify the manufacture of the extractor apparatus.

The apparatus may comprise an injector system and a support system for supporting the enclosures, and at least one of the injector system and the support system may be arranged in such a manner as to make it possible to select the enclosures in which the fluid is to be injected.

By way of example, selecting the enclosure(s) in which the fluid is injected is performed by a controller for controlling the flow of fluid in each of the injectors.

In a variant or in addition, selection can be performed by positioning the injectors adequately relative to the enclosures.

The enclosures may be fitted on the extractor apparatus or they may belong thereto.

The enclosures may optionally belong to a single refill. The refill may comprise a cartridge containing a plurality of enclosures. The refill may also comprise a plurality of capsules that are secured to one another, at least during use, defining respective different enclosures.

The support system may be arranged to enable the enclosures to be moved relative to the injector system. This movement may be performed manually or it may be motor-driven, it may be in rotation, or in translation, or in some other manner, e.g. in such a manner as to cause at least one injector to penetrate into a selected enclosure.

As mentioned above, the extractor apparatus may include a heater system to raise the temperature of the fluid to greater than 80° C. prior to injection.

When the enclosures include coloring agents, the extractor apparatus may include a selector system arranged firstly to enable a user to select one tint from a plurality of tints, and secondly to confirm injection of the fluid so as to collect a cosmetic composition of tint that corresponds to the selected tint. By way of example, the selector system may act on the controller for controlling the flow of fluid in the various injectors so that predefined quantities of fluid pass through the various enclosures.

The selector system may include a micro-computer or any other computer system.

In another of its aspects, the invention also provides an extractor apparatus enabling a fluid to flow, and in particular to percolate, through at least one enclosure in order to prepare a cosmetic composition, the apparatus comprising:

an injector system comprising a plurality of injectors for injecting at least one fluid into a plurality of enclosures;

a support system for supporting the plurality of enclosures; and

a collector system for collecting solutions that come from fluid flowing through the enclosures, the system being arranged to mix the solutions.

By way of example, the collector system comprises a receptacle placed under the enclosures and/or channels enabling the flow of liquid to be guided after it has passed through the enclosures.

The plurality of enclosures may belong to the same refill, the enclosures being contained in a cartridge, for example.

At least two enclosures may be separated by a partition inside the refill.

The enclosures may be distributed angularly about an axis of the refill, or they may be in alignment, or they may be disposed circularly, or in some other way.

The refill may comprise a body made at least in part by molding a thermoplastic material, or by stamping and/or folding or thermoforming a material in sheet form.

The plurality of enclosures may also be defined by a plurality of distinct capsules that are possibly connected to one another.

The capsules may be carried by a support element, which support element may be annular or elongate in shape, for example.

The capsules may be placed in rows, or otherwise.

Each enclosure may be associated with at least one sealing gasket arranged to co-operate with at least one injector. By way of example, the sealing gasket may be a membrane of an elastically deformable material that is arranged to allow the injector to pass therethrough.

Each enclosure may be substantially airtight.

The refill may include keying means so as to enable it to be placed on the extractor apparatus in a predefined orientation.

As mentioned above, at least one of the substances contained in the enclosures may comprise a coloring agent for coloring keratinous materials. Specifically, the plurality of enclosures may comprise a plurality of coloring agents of different colors for coloring keratinous materials, so as to make it possible to prepare a cosmetic composition having a personalized tint, for example.

Where appropriate, the compounds contained in the various enclosures may all be different.

The substances contained in the enclosures are advantageously in powder form, or in a variant in the form of a gel, a paste, or a liquid.

Amongst the substances that may be contained in the various enclosures secured to the extractor apparatus, e.g. belonging to a common refill, the following combinations are possible, amongst others:

a first substance comprising a first coloring agent contained in a first enclosure, and a second substance comprising a second coloring agent, different from the first, and contained in a second enclosure;

a first substance comprising a first cosmetically or dermatologically active agent contained in a first enclosure, and a second substance comprising a second cosmetically or dermatologically active agent, different from the first, and contained in a second enclosure; and

a first substance comprising a first plant or mineral extract contained in a first enclosure, and a second substance comprising a second plant or mineral extract, different from the first, and contained in a second enclosure.

### BRIEF DESCRIPTION OF THE DRAWINGS

Various details of the present invention may be better understood on reading the following detailed description of non-limiting embodiments, and on examining the accompanying drawings in which:

FIG. 1 is a diagrammatic perspective view of an example of a refill;

FIG. 2 is a cross-sectional view of the FIG. 1 refill;

FIG. 3 is a plan view taken along III-III of FIG. 2;

FIG. 4 is a view analogous to FIG. 3 showing a variant embodiment;

FIG. 5 is a view analogous to FIG. 2 showing a variant embodiment;

FIG. 6 is a fragmentary perspective view showing an example of an extractor apparatus suitable for being used in preparing the cosmetic composition;

FIG. 7 is a fragmentary cross-sectional view of the refill in place in the extractor apparatus;

FIG. 8 shows the positioning of the injectors relative to the compartments;

FIG. 9 is a simplified diagrammatic view of a feed circuit of an extractor apparatus;

FIG. 10 is a perspective view of another example of a refill showing the corresponding positioning of the injectors;

FIG. 11 is a cross-sectional view taken along XI-XI of FIG. 10;

FIG. 12 is a plan view of another example of a refill;

FIG. 13 is a cross sectional view taken along XIII-XIII of FIG. 12;

FIG. 14 is a plan view of another example of a refill; and

FIG. 15 is a diagrammatic fragmentary view of another way in which the various injectors can be positioned relative to a plurality of independent enclosures.

### DETAILED DESCRIPTION OF EMBODIMENTS

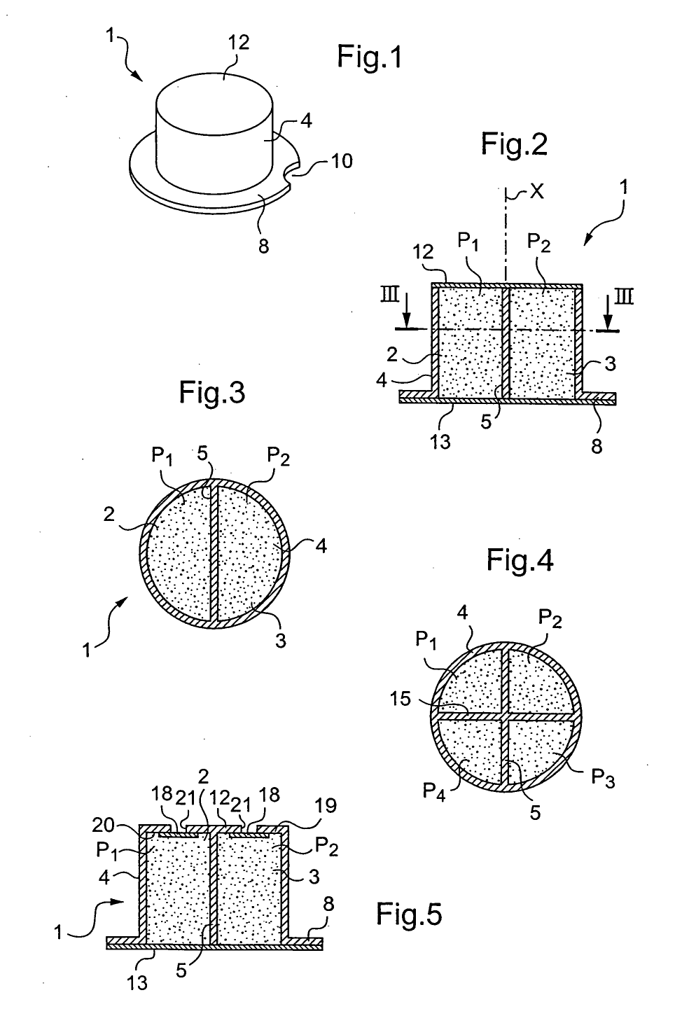

FIGS. 1 to 3 show a refill 1 constituting an embodiment of the invention, presented in the form of a cartridge. The word “refill” is used because a user could refill (replenish) the user's supply of the substance P by purchasing one or more refills 1 when desired (for example, when the user's supply of substance P is depleted). The refills 1 are not themselves necessarily refilled with substance P (that is, they are not necessarily re-used), although in embodiments they could be refilled with substance P.

By way of example, the refill 1 contains at least two substances P₁ and P₂ respectively contained in separate manner in at least two enclosures 2 and 3 defined by the refill.

In an example, the enclosures 2 and 3 are formed by two compartments of a tubular body 4 that are separated by a single partition 5. By way of example, the partition is made by being molded as a single piece together with the body 4.

In the example shown, the body 4 is made with a flange 8 at one end, and by way of example it is generally cylindrical about an axis X.

The flange 8 may be provided with keying means 10, e.g. in the form of a recessed or projecting portion in relief, e.g. a notch.

Alternatively, if the body 4 is made without keying means 10, it could be made to be of a shape that is not circularly symmetrical, so as to allow it to be positioned in the associated extractor apparatus in one way only.

The body 4 is closed on top by a first wall 12, and at the bottom by a second wall 13.

By way of example, the walls 12 and 13 are constituted by film which is heat sealed to the body 4 and the partition 5 in such a manner as to close the enclosures 2 and 3 in airtight manner. By way of example, the film may include at least one layer of metal, in particular of aluminum.

The volume of each of the enclosures 2 and 3 is preferably less than or equal to 25 cm³, and more preferably less than or equal to 10 cm³. The volume of each enclosure 2 and 3 may also be in the range of about 1 cm³ to about 5 cm³. The volume of each enclosure 2 and 3 may be about the same or different from each other.

Naturally, various modifications could be made to the shape of the refill 1 without going beyond the ambit of the present invention.

The number of enclosures contained in the refill 1 may be other than two, in particular, for example it could lie in the range 2 to 10, e.g. being four as shown in FIG. 4.

FIG. 4 shows that the refill 1 may have at least one second partition 15 co-operating with the partition 5 to define four enclosures, thus making it possible to contain two additional substances P₃ and P₄ separately from the substances P₁ and P₂.

Where appropriate, the refill 1 may have a first wall 12 which is made at least in part integrally with the body 4, e.g. by molding.

By way of example, the first wall 12 may then be pierced by openings 21 associated respectively with the different enclosures, as shown in FIG. 5.

The openings 21 may be closed in such a manner as to enable the injectors of the associated extractor apparatus to enter therethrough.

By way of example, closing may be performed by means of diaphragm 18 heat sealed to the inside face 20 of the first wall 12. In a variant, the diaphragms 18 are secured to the outside on the first wall 12 or they are injected thereover, e.g. using an elastomer material. The diaphragm 18 may also be made integrally with the first wall 12 and out of the same material, by giving the first wall 12 reduced thickness locally, or by making zones of weakness. While injection is taking place, the diaphragm 18 may be arranged to be perforated or to become detached, at least in part, from the first wall 12.

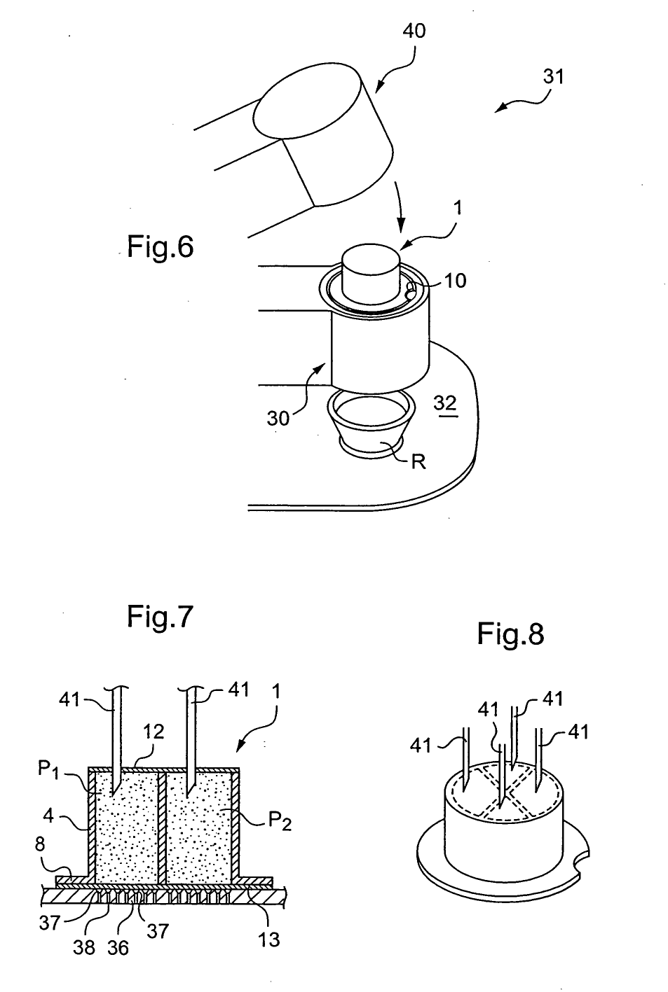

In order to use the refill 1 of FIG. 4, the user may place it on a support system 30 of an extractor apparatus 31 that also includes an injector system 40, as shown in FIG. 6.

In the example shown, the extractor apparatus 31 includes a gap 32 under the support system 30 for receiving a receptacle R suitable for collecting a solution coming from percolation of a fluid through the refill enclosure(s) selected by the user.

As shown in FIG. 7, the support system 30 can include a grid 36 provided with portions in relief 37 against which the second wall 13 can be pressed under the pressure of the fluid injected into the selected enclosure.

The second wall 13 is arranged to be perforated by the portions in relief 37 of the grid, thereby enabling the injected fluid to flow through the channels 38 in the grid 36 towards the receptacle R.

The second wall 13 may advantageously be opened in such a manner as substantially to retain inside the enclosure in question any particles of the substance that have not dissolved.

The receptacle R may be wide enough to collect the solutions flowing from the various enclosures, given their positions. Where appropriate, a system for collecting the solutions may be provided to convey them into the receptacle when the enclosures are not situated vertically thereover.

As can be seen in FIGS. 7 and 8, the injector system 40 includes a plurality of injectors 41 capable of passing through the first wall 12 and of penetrating into the corresponding enclosures.

The extractor apparatus 31 may include sealing means (not shown) making it possible, where necessary, to guarantee that each injector 41 penetrates in leaktight manner into the corresponding enclosure.

By way of example, these sealing means may comprise at least one sealing gasket that bears against the outside of the refill, at least during injection.

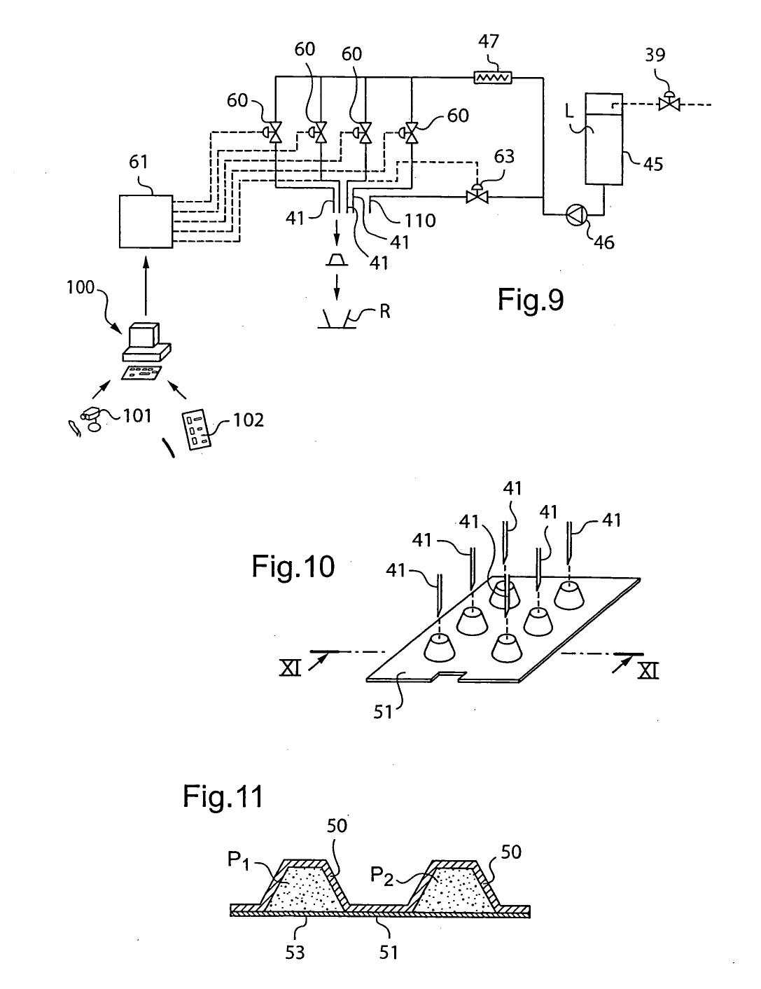

As shown diagrammatically in FIG. 9 the extractor apparatus 31 may include a feed circuit for feeding the injectors 41 with extractor fluid, the feed circuit having: at least one tank 45 for containing the liquid L that is to be injected; at least one pump 46; and at least one heater member 47 serving to raise the liquid L to the desired temperature, and where appropriate, to generate steam. This can contribute to generating the desired injection pressure.

In the example described, the fluid injection pressure is, for example, at least 3 bars and preferably at least 10 bars, and the injection temperature is at least 80° C., for example. In particular, the temperature of the fluid may be sufficient for the fluid to present both liquid and gaseous phases while it is being injected into the selected enclosure.

The tank 45 of the extractor apparatus may be filled manually or automatically, being connected to a water pipe, e.g. via a solenoid valve 39.

Where appropriate, the tank 45 may be removable so as to make it easier to fill manually.

The heater member 47 may comprise an electrical resistor element.

As shown in FIG. 9, the extractor apparatus may have a plurality of flow-control members 60, each associated with a respective injector 41 and controlled in common by a control system 61 in such a manner that only the selected injector(s) 41 is/are fed by the fluid for injection, for example.

The quantity of fluid percolating in the selected enclosure(s) may be predefined, or, for example, may be selected as a function of the concentration desired for the or each compound extracted by percolation.

By way of example, the flow-control members 60 may be proportional solenoid valves enabling the flow through each injector to be adjusted in a manner other than by on/off adjustment alone. For example, the control members 60 may be piezoelectric. The flow can also be adjusted by on/off adjustment.

By adjusting the different flow rates, several shades can be obtained, for example.

For example, the user may select at least two enclosures enabling a particular color shade or coloring agent to be obtained by mixing the extraction solutions.

By way of example, the injectors 41 are arranged in such a manner that all of them penetrate into the enclosures associated with the refill, as shown in FIG. 7, with fluid being injected into only the selected enclosure(s) by controlling the flow.

In a variant, the injector system is arranged in such a manner that the injectors 41 and/or the enclosures can be moved relative to one another so that only the injector(s) 41 that is/are being fed penetrate(s) into the corresponding enclosure(s). By way of example, the pressure of the injection fluid can contribute to moving injector while it is being fed, so as to cause it to penetrate into the corresponding enclosure.

FIG. 9 also shows the possibility of the feed circuit exchanging data with a computer system 100, e.g. comprising at least one microcomputer, a personal digital assistant (PDA), a terminal connected to a network, or a mobile telephone, or even that is integrated in the extractor apparatus.

By way of example, the computer system 100 may be associated with a data acquisition system, e.g. comprising a camera 101 or any other device for analyzing keratinous materials, e.g. for analyzing the color of the skin or of the hair.

The computer system 100 may also be associated with a color chart or with a hair color chart 102 or with some other evaluation tool, e.g. a questionnaire.

The computer system 100 may be arranged in such a manner as to enable a user to select at least one characteristic of the cosmetic composition that is to be prepared by means of the extractor apparatus and the enclosures containing the various substances. By way of example, this characteristic may be color.

The computer system 100 may be used to enable the user to select a desired tint, and the computer system 100 may be arranged to send data to the control system 61 enabling it to control the injection of fluid into enclosures that have been predefined in such a manner as to obtain a composition having the desired properties.

As a function of the substances contained in the enclosures, the user may also select a characteristic for the composition in addition to color or to dyeing capacity, for example a fragrance, a consistency, one or more active ingredients, a duration for its activity, a dilution, or a characteristic enabling the composition to be adapted to a particular type of skin or hair, amongst others.

Where appropriate, the extractor apparatus may include an output 110 enabling the liquid L to be dispensed into the receptacle R without passing through the enclosures, e.g. for the purpose of diluting the solution(s) obtained by percolation into the receptacle R so as to obtain a predefined volume of composition.

The output 110 may be fed by means of a solenoid valve 63 or any other means for controlling the quantity of fluid that is dispensed, such as a pump, for example.

By way of example, the solenoid valve 63 is controlled by the control system 61, so as to have a volume of composition in the receptacle R that corresponds to a predefined value, as a function of the quantities already dispensed after injection into the selected enclosure(s).

The extractor apparatus 31 may include one or more check valves and/or solenoid valves (not shown), together with one or more sensors for sensing level, weight, pressure, and/or temperature, together with a control system for the various electrical, electromechanical, and sensor members.

By way of example, a level sensor can be useful for detecting when it is necessary to refill the tank 45 or to enable the receptacle R to be filled with a predefined total quantity of composition.

A weight sensor may be useful for determining the quantity of composition that has been delivered to the receptacle R, for example.

Where appropriate, the person skilled in the art can easily refer to the extractor apparatuses used for preparing beverages of the “espresso” type, and the feed circuits thereof can be reproduced, at least in part.

By way of example, such apparatuses are disclosed in the following publications: AT 168 405, U.S. Pat. No. 2,688,911, DE 3 243 870, IT 1 265 636, and WO 2004/006 740-A2.

The refill 1 may be in a form other than that of a cartridge, and for example as shown in FIGS. 10 and 11, it may have enclosures defined by capsules 50 carried by a support element 51, e.g. in the form of a plate.

By way of example, the capsules 50 may have a first wall that is formed by stamping or thermoforming a sheet material, e.g. aluminum or a laminate including aluminum. As stamped or thermoformed, this wall may define the support element 51 around the capsules 50.

The capsules 50 may be closed by a second wall 53, e.g. constituted by a film that is heat sealed on the support element 51.

The support element 51 may include keying means 56, e.g. the form of a notch.

Where appropriate, the support element 51 and the capsules 50 may be made separately.

For example, the support element 51 can be made in the form of a plate with holes, and the capsules can be secured in the holes, e.g. as a function of the substances that it is desired to associate within a single refill.

The capsules may be fastened in the holes of the support element in optionally releasable manner. Where appropriate, releasable fastening can enable the user to replace used capsules with new capsules. By way of example the releasable fastening may be performed by friction and/or by snap-fastening.

In the example of FIG. 10, the capsules 50 are disposed in rows.

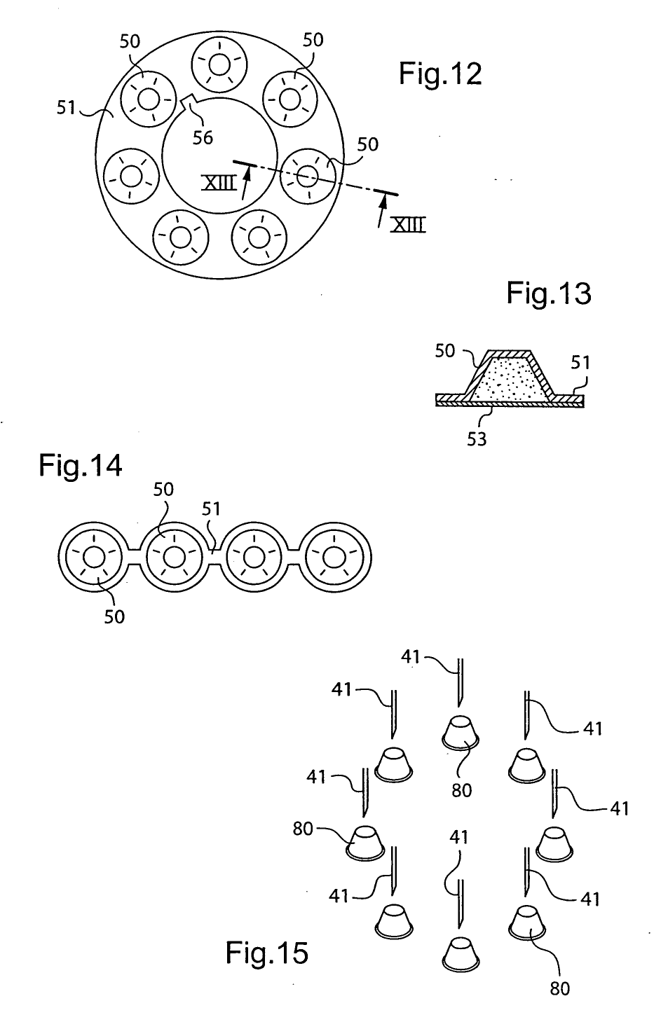

In the variant of FIGS. 12 and 13, the support element 51 is annular in shape, and in the example of FIG. 14, it is in the form of a strip.

In the variant shown in FIG. 15, the enclosures are defined by independent capsules 80, and the extractor apparatus has a plurality of injectors respectively associated with the various capsules.

The support system of the extractor apparatus includes a variety of housings for receiving the capsules, for example.

Injection may be performed into a selected capsule by causing the injector to penetrate into the corresponding enclosure, e.g. by moving the injector and/or the capsule, where this movement may be caused, the example, by the user acting on at least one of the injector system and the support system. This movement may also be the result of drive from a pneumatic or a hydraulic motor, e.g. with assistance from the pressure of the fluid.

The invention is not limited to the embodiments shown.

In particular, the refill may be given further shapes other than those shown, the example in the form of a small tray, a sachet, etc. Capsules or sachets containing different substances may be contained in a single package.

The enclosures may be filled at the time of manufacture. In a variant, the enclosures may be filled as a function of requests made by a consumer, for example.

Different compartments in a single refill may be made separately and then assembled together.

During manufacture, the enclosures may also be in a deployed configuration, and this configuration can be modified during manufacture and/or in use.

Fluid may flow in a selected enclosure either vertically or otherwise.

In the examples shown, injection into an enclosure takes place via a single injector that is associated with the enclosure, however in a variant injection may take place via a plurality of injectors, e.g. in order to improve putting the fluid into contact with the substance contained in the enclosure, or to enable the substance to be put into contact with a different fluid.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

The term “comprising a” should be understood as being synonymous with “comprising at least one”, unless specified to the contrary.

What is claimed is:

1. A method of preparing a cosmetic composition, the method comprising:
injecting into a plurality of enclosures containing different substances at least one fluid through a plurality of injectors so as to produce a plurality of solutions which, after mixing, form all or part of the cosmetic composition.

2. A method according to claim 1, wherein the fluid is injected into the plurality of enclosures through injectors that are respectively associated with different enclosures of the plurality of enclosures.

3. A method according to claim 1, the plurality of enclosures belonging to a common refill.

4. A method according to claim 3, the plurality of enclosures being defined by compartments of a common cartridge.

5. A method according to claim 1, the plurality of enclosures not belonging to a common refill.

6. A method according to claim 1, with injection into the plurality of enclosures taking place simultaneously.

7. A method according to claim 1, with injection into the plurality of enclosures taking place sequentially.

8. A method according to claim 1, the fluid being injected under pressure and the enclosures being defined at least in part by a wall that is configured for opening under the effect of the pressure of the fluid.

9. A method according to claim 1, at least one of the enclosures containing a coloring agent for coloring keratinous material.

10. A method according to claim 1, the quantity of fluid that is injected into at least one enclosure being controlled by at least one control element that is controlled electrically.

11. A method according to claim 1, the injection of fluid into the plurality of enclosures being performed so as to have a predefined volume of cosmetic composition.

12. A method according to claim 1, the enclosures being defined at least in part by a perforatable wall, and the injectors being arranged to perforate said wall.

13. A method according to claim 1, comprising selecting at least one desired property of the cosmetic composition or at least one level for a given property of the cosmetic composition, and controlling the injection of the fluid in such a manner as to obtain the selected property and/or the at least one level.

14. A method according to claim 13, the selected property relating to a color of the composition or to a dyeing capacity of the composition.

15. A method according to claim 1, the solutions -being collected in a receptacle.

16. A method according to claim 15, the receptacle containing at least one compound for mixing with the solutions.

17. A method according to claim 1, the fluid including water.

18. A method according to claim 17, the fluid being water.

19. A method according to claim 1, the temperature of the fluid being greater than 80° C.

20. A method according to claim 1, the injected fluid being in the form of both vapor and liquid phases.

21. A method according to claim 1, the pressure of the injected fluid being greater than or equal to 1 bar.

22. A method according to claim 21, wherein the pressure is greater than or equal to 3 bars.

23. A method according to claim 21, wherein the pressure is greater than or equal to 10 bars.

24. A method according to claim 1, the solutions obtained from the extraction being received in a receptacle.

25. An extractor apparatus enabling at least one fluid to flow through a plurality of enclosures in order to prepare a cosmetic composition, the apparatus comprising:
an injector system comprising a plurality of injectors for injecting at least one fluid into a plurality of enclosures;
a support system for supporting the plurality of enclosures; and
a collector system for collecting solutions that come from fluid flowing through the enclosures, the collector system being configured to mix the solutions.

26. An apparatus according to claim 25, further comprising a selector for selecting at least one of the enclosures in which the injection of fluid takes place.

27. An apparatus according to claim 25, further comprising a controller for controlling the quantity of fluid that is delivered by each of the injectors.

28. An apparatus according to claim 27, further comprising a control system for controlling the controller as a function of at least one sought-after property of the composition.

29. An apparatus according to claim 28, wherein the property is tint.

30. An apparatus according to claim 25, the injected fluid being under a pressure of at least 3 bars and at a temperature greater than 80° C.

31. An apparatus according to claim 25, the fluid being in the form of both vapor and liquid phases.

32. An apparatus according to claim 25 , the fluid being water.

33. An assembly enabling a cosmetic composition to be prepared, the assembly comprising:
a plurality of enclosures containing different cosmetic substances; and
an extractor apparatus comprising a plurality of injectors in order to inject a fluid in at least one selected enclosure and collect at least one solution by the fluid flowing through the at least one selected enclosure.

34. An assembly according to claim 33, the plurality of enclosures belonging to a common refill.

35. An assembly according to claim 34, the refill including a plurality of capsules secured to one another and respectively defining different enclosures.

36. An assembly according to claim 33, the extractor apparatus including a heater system to raise the temperature of the fluid to greater than 80° C. prior to injection.

37. An assembly according to claim 33, the extractor apparatus being arranged to raise the pressure of the injected fluid to at least 1 bar.

38. An assembly according to claim 33, the extractor apparatus including a selector system configured to enable a user to select one tint amongst a plurality of tints, and configured to control the injection of the fluid so as to collect a cosmetic composition of tint that corresponds to the selected tint.
